(19) United States
(12) Patent Application Publication
(10) Pub. No.: US 2025/0057824 A1
(43) Pub. Date: Feb. 20, 2025

(54) NOSE-TO-BRAIN DELIVERY FORMULATION OF NIMODIPINE, PREPARATION AND USE

(71) Applicant: Nasal-Phyto (Shanghai) Pharmaceutical Technology Co., Ltd., Shanghai (CN)

(72) Inventors: Jingyi Liu, Shanghai (CN); Qing You, Shanghai (CN); Chengwei Wang, Shanghai (CN)

(21) Appl. No.: 18/482,835

(22) Filed: Oct. 6, 2023

(30) Foreign Application Priority Data
Aug. 17, 2023 (CN) 202311045174.5

(51) Int. Cl. A61K 31/4422; A61K 9/00; A61K 47/10; A61K 47/12; A61K 47/14; A61K 47/22; A61K 47/24; A61K 47/44

(52) U.S. Cl. CPC A61K 31/4422; A61K 9/0043; A61K 47/10; A61K 47/12; A61K 47/14; A61K 47/22; A61K 47/24; A61K 47/44

(57) ABSTRACT

The present disclosure provides a nose-to-brain delivery formulation of nimodipine. The present disclosure also provides a method of preventing or treating ischemic disorders in a patient, in particular cerebral ischemic disorders, cerebral vasospasm due to subarachnoid hemorrhage, and sudden deafness, comprising administering said nimodipine nose-to-brain delivery formulation. The present disclosure also provides a composition, comprising a nose-to-brain delivery formulation of nimodipine and pharmaceutically acceptable excipient.

### FIELD

The present disclosure belongs to the field of pharmaceuticals, and relates to a nose-to-brain delivery formulation of a drug, in particular relates to a high safety, brain-targeted nose-to-brain delivery formulation, specifically relates to a high safety, brain-targeted nose-to-brain delivery formulation of nimodipine.

### BACKGROUND

Nimodipine (NM) is the second generation of dihydropyridine calcium channel antagonist, which can inhibit calcium influx in cerebral vascular smooth muscle cells and cerebral neuronal cells. The drug acts preferentially on small arteries, which can selectively dilate cerebral blood vessels, increase cerebral blood flow, protect ischemic neuronal cells, promote memory and facilitate intellectual recovery, and have both neurological and psycho-pharmacological effects. Clinically used in the treatment of cerebral ischemic diseases, cerebral vasospasm caused by subarachnoid hemorrhage, sudden deafness and other diseases, Nimodipine achieved better efficacy. However, the water solubility of nimodipine is extremely poor, the oral first-pass effect is strong, the bio-availability is low, and the clinically used injections use a large amount of ethanol as a solvent, resulting in high irritation. Nimodipine also has cardiovascular toxicity and hepatotoxicity. In addition, the blood-brain barrier is the most important bottleneck for the therapeutic effect of drugs in central diseases. Nimodipine is not effective in crossing the blood-brain barrier, resulting in a low brain exposure, and can not play a therapeutic role in brain diseases to the maximum extent.

Nose-to-brain delivery system is mainly for upper nasal tract drug delivery, as a non-invasive drug delivery, it has received wide attention in recent years. The mucosa of the olfactory region of the upper nasal tract has many microscopic villi, which can greatly increase the effective area of drug absorption. Nose-to-brain delivery has the following advantages: \textcircled{1} Convenient drug administration, and it is desirable for patients to use the drug on their own; \textcircled{2} Rapid absorption of the drug and fast onset of action; \textcircled{3} Avoidance of hepatic first-pass effect; and \textcircled{4} Increase in the delivery of the drug to the brain. Therefore, nasal drug delivery is expected to increase the drug concentration level in the brain, enhance the therapeutic effect of the drug and reduce the toxic side effects.

In clinical practice, it has been found that although the olfactory nerve pathway in the upper nasal olfactory region is a noninvasive and convenient route for central drug delivery, drug delivery to the upper nasal olfactory region is limited by many physiological factors. Conventional drug delivery devices are unable to effectively deliver drugs to the nasal olfactory region site, thus making the purpose of drug nose-to-brain delivery unattainable. Especially with the help of animal models for the indirect study of human diseases, small animals as the most commonly used and universal mammalian model carriers, but due to their own small size, many devices and appliances are not suitable for application to smaller size animals, resulting in a certain obstacle to the establishment of animal models.

Because nimodipine has a low solubility of 0.002 mg/mL in water, it is generally solubilized with alcohol-based preparations for clinical use. Currently, studies on the formulation of nimodipine have focused on reducing the amount of ethanol used, but have not reduced cardio-toxicity or hepatotoxicity.

Currently, commercially available nimotop injection is prone to sub-injection irritation and thrombophlebitis due to the use of up to 20% ethanol.

In prior art, nimodipine nanoemulsion injections have been developed. The formulations mentioned above have no longer use ethanol, with reducing irritation under the injection route and the probability of thrombophlebitis, they nevertheless have a low brain volume entry, a high blood level and a high risk of cardiotoxicity and hepatotoxicity.

The prior art also developed a nimodipine gels for transnasal administration. However, these gels are applied to the lower nasal tract, and the improvement is still to increase the amount of drug in the bloodstream, and when the formulation is applied to the upper nasal tract, it has a very low permeability coefficient that makes it difficult to enter the brain through the nose. Since nimodipine works by entering the brain or cerebrospinal fluid, these formulations do not improve the efficacy of the drug and the risk of cardiotoxicity and hepatotoxicity remains high because of the still high level of the drug in the bloodstream.

In addition, current studies of nasal mucosal delivery systems have found that such formulations can cause irritation of the nasal mucosa, mainly ciliotoxic effects; including the effects of drugs and excipients on ciliary activity.

Therefore, how to ensure that nimodipine have both good nasal mucosal absorption performance and nasal safety, as well as high brain entry and having a high cerebral to blood ratio, is the key to whether nimodipine can be developed as a nasal upper-nasal tract drug delivery formulation.

### SUMMARY

The present disclosure provides a nimodipine nose-to-brain delivery formulation that has a high cerebrospinal fluid, high olfactory bulb and highcortical brain tissue concentration, is non-nasally toxic and irritating, transmits through the mucous membrane of the olfactory region of the upper nasal tract directly into the brain tissue, and can be smoothly absorbed with a high cerebral to blood ratio and has a high cerebrospinal fluid to blood ratio.

According to the first aspect of the present disclosure, the present disclosure provides a nose-to-brain delivery formulation of nimodipine comprising nimodipine, a solvent, a diluent and an absorption enhancer, wherein the solvent is diethylene glycol monoethyl ether, and the absorption enhancer is one or more substances selected from phospholipids, n-dodecyl-β-D-maltoside (DDM), cyclopentadecanolide, Polyoxyl 15 Hydroxystearate (HS15), and oleoyl polyoxyl-6 glycerides.

According to the second aspect of the present disclosure, the present disclosure provides a method for preparing the nose-to-brain delivery formulation of nimodipine, comprising: adding the nimodipine and the solvent into a glass-bottle, preferably by sonication to dissolve the nimodipine; and adding the absorption enhancer, preferably by shaking and sonication to dissolve the absorption enhancer; and, optionally, adding an aromatizer, and finally, using a diluent to the total mass of each formulation.

According to the third aspect of the present disclosure, the present disclosure provides the nose-to-brain delivery formulation of nimodipine obtained by the above method of preparing the nose-to-brain delivery formulation of nimodipine.

According to the forth aspect of the present disclosure, the present disclosure provides a method of preventing or treating ischemic disorder in a patient, in particular cerebral ischemic disorders, cerebral vasospasm caused by subarachnoid hemorrhage, and sudden deafness, comprising the step of administering a therapeutically effective amount of the nose-to-brain delivery formulation of nimodipine to a patient in need thereof.

According to the fifth aspect of the present disclosure, the present disclosure provides a use of the nose-to-brain delivery formulation of nimodipine in treating ischemic disorder by means of transnasal entry into the brain, in particular the use of treating cerebral ischemic disorders, cerebral vasospasm caused by subarachnoid hemorrhage, and sudden deafness. Especially, the present disclosure also provides a use of the above mentioned nose-to-brain delivery formulation of nimodipine in preparing for treating ischemic disorder by means of transnasal entry into the brain, in particular the use of treating cerebral ischemic disorders, cerebral vasospasm caused by subarachnoid hemorrhage, and sudden deafness.

According to the sixth aspect of the present disclosure, the present disclosure provides a composition, comprising the nose-to-brain delivery formulation of nimodipine and any suitable pharmaceutically acceptable excipient.

According to the seventh aspect of the present disclosure, the present disclosure provides a use of the combination of a solvent, a diluent and an absorption enhancer in preparing of nose-to-brain delivery formulation of nimodipine, wherein the solvent is diethylene glycol monoethyl ether, and the absorption enhancer is one or more substances selected from phospholipids, n-dodecyl-β-D-maltoside (DDM), cyclopentadecanolide, polyoxyl 15 hydroxystearate (HS15), and oleoyl polyoxyl-6 glycerides.

According to another aspect of the present disclosure, the present disclosure provides a method for preventing or treating an ischemic disorder in a patient comprising administering to a subject in need thereof a therapeutically effective amount of a nose-to-brain delivery formulation of nimodipine, the nose-to-brain delivery formulation comprising nimodipine, a solvent, a diluent and an absorption enhancer, wherein the solvent is diethylene glycol monoethyl ether, and the absorption enhancer is one or more substances selected from phospholipids, n-dodecyl-β-D-maltoside, cyclopentadecanolide, Polyoxyl 15 Hydroxystearate, and oleoyl polyoxyl-6 glycerides.

According to another aspect of the present disclosure, the present disclosure provides a composition, comprising a nose-to-brain delivery formulation of nimodipine and pharmaceutically acceptable excipient, the nose-to-brain delivery formulation comprising nimodipine, a solvent, a diluent and an absorption enhancer, wherein the solvent is diethylene glycol monoethyl ether, and the absorption enhancer is one or more substances selected from phospholipids, n-dodecyl-β-D-maltoside, cyclopentadecanolide, Polyoxyl 15 Hydroxystearate, and oleoyl polyoxyl-6 glycerides.

### BRIEF DESCRIPTION OF DRAWINGS

Other features, objects and advantages of the present disclosure will become more apparent by the detailed description of the non-limiting embodiments with reference to the following accompanying drawings:

FIG. 1 is a graph of a comparison of drug concentrations in plasma after administration of the nose-to-brain delivery formulations of formulations 5-8 of Embodiment 5 of the present disclosure to the olfactory region of the upper nasal tract of mice and after commercially available Nimotop® injection were injected intravenously into mice in Pharmacologic Embodiment 1.

FIG. 2 is a graph of a comparison of drug concentrations in cerebrospinal fluid after administration of the nose-to-brain delivery formulations of formulations 5-8 of Embodiment 5 of the present disclosure to the olfactory region of the upper nasal tract of mice and after commercially available Nimotop® injection were injected intravenously into mice in pharmacological embodiment 1.

FIG. 3 is a graph of a comparison of drug concentrations in olfactory bulb after administration of the nose-to-brain delivery formulations of formulations 5-8 of Embodiment 5 of the present disclosure to the olfactory region of the upper nasal tract of mice and after commercially available Nimotop® injection were injected intravenously into mice in pharmacological Embodiment 1.

FIG. 4 is a graph of a comparison of drug concentrations in cortical brain tissue after administration of the nose-to-brain delivery formulations of formulations 5-8 of Embodiment 5 of the present disclosure to the olfactory region of the upper nasal tract of mice and after commercially available Nimotop® injection were injected intravenously into mice in pharmacological Embodiment 1.

FIG. 5 is a graph of a comparison of drug concentrations in cerebrum after administration of the nose-to-brain delivery formulations of formulations ST202-Y-land ST202-Y-3 of the present disclosure to the olfactory region of the upper nasal tract of mice and after commercially available Nimotop® tablet were injected intravenously into mice in pharmacological Embodiment 2.

### DETAILED DESCRIPTION

Aiming at the technical problems in the prior art that the nimodipine has a poor brain transmission rate, and the drug needs to pass the blood-brain barrier to enter the brain after being administered by the mucous membrane of the lower nasal tract and intravenously, which results in a low concentration of the drug in the brain and the peripheral circulatory system of nimodipine prone to cause the side effects of cardiovascular toxicity and hepatotoxicity, the present disclosure provides a reservoir-type nose-to-brain delivery formulation of nimodipine of high cerebrospinal fluid, high olfactory bulb and high cortical brain tissue concentration, free from local irritation in nasal cavity, and can be present in the long term and stably, transmits the mucous membrane of the olfactory region of the upper nasal tract to enter the brain tissue directly, and can be absorbed smoothly, and has a high cerebral-to-blood ratio and a high cerebrospinal fluid-to-blood ratio.

According to the first aspect of the present disclosure, the present disclosure provides a nose-to-brain delivery formulation of nimodipine comprising nimodipine, a solvent, a diluent and an absorption enhancer, wherein the solvent is diethylene glycol monoethyl ether, and the absorption enhancer is one or more substances selected from phospholipids, n-dodecyl-β-D-maltoside, cyclopentadecanolide, polyoxyl 15 hydroxystearate, and oleoyl polyoxyl-6 glycerides.

In some embodiments, the absorption enhancer can be a phospholipid.

The nose-to-brain delivery formulation of nimodipine in this disclosure, the diluent can be one or more species selected from isopropyl myristate, ethyl oleate, medium chain triglycerides, castor oil, soybean oil, sesame oil, oleic acid, vitamin E, glyceryl monolinoleate, propylene glycol laurate, or propylene glycol dicaprylate/dicaprate, and more preferably the diluents comprising a combination of one or more of ethyl oleate, isopropyl myristate, and propylene glycol dicaprylate/dicaprate.

The nose-to-brain delivery formulation of nimodipine in this disclosure, the phospholipid can be lecithin or soya lecithin, Further, the lecithin may be of any model number, for example it may be lecithin E80 or lecithin PL100M, and the soya lecithin may be of any model number, for example it may be soya lecithin S75 or soya lecithin S100.

The nose-to-brain delivery formulation of nimodipine in this disclosure, the nimodipine is available in 4-50 parts by weight, preferably, 5-40 parts by weight, for example, it may be 5, 10, 15, 20, 30, 40 parts by weight, most preferably, 15 parts by weight.

The nose-to-brain delivery formulation of nimodipine in this disclosure, the solvent is available in 5-300 parts by weight, preferably, 40-200 parts by weight, for example, it may be 40, 50, 80, 100, 120, 150, 160, 200 parts by weight.

The nose-to-brain delivery formulation of nimodipine in this disclosure, the diluent is available in 500-900 parts by weight, preferably, 700-850 parts by weight, for example, it may be 725, 750, 785, 805, 825, 850 parts by weight.

The nose-to-brain delivery formulation of nimodipine in this disclosure, the absorption enhancer is available in 20-200 parts by weight, preferably, 40-160 parts by weight, for example, it may be 40 parts by weight, 50 parts by weight, 60 parts by weight, 80 parts by weight, 100 parts by weight, 120 parts by weight, 150 parts by weight, 160 parts by weight.

The nose-to-brain delivery formulation of nimodipine in this disclosure is in the form of a solution.

In some embodiments of the present disclosure, the nose-to-brain delivery formulation of nimodipine contains the nimodipine, the solvent, the diluent and the absorption enhancer.

In some embodiments of the present disclosure, the nose-to-brain delivery formulation of nimodipine contains 5-40 parts by weight nimodipine, 40-200 parts by weight diethylene glycol monoethyl ether, 40-160 parts by weight phospholipid, 700-850 parts by weight isopropyl myristate, preferably, the phospholipid can be lecithin or soya lecithin, wherein the lecithin may be of any model number, for example it may be lecithin E80 or lecithin PL100M, and the soya lecithin may be of any model number, for example it may be soya lecithin S75 or soya lecithin S100.

In some embodiments of the present disclosure, the nose-to-brain delivery formulation of nimodipine contains 15 parts by weight nimodipine, 100 parts by weight diethylene glycol monoethyl ether, 100 parts by weight phospholipid, 785 parts by weight isopropyl myristate, preferably, the phospholipid can be lecithin or soya lecithin, wherein the lecithin may be of any model number, for example it may be lecithin E80 or lecithin PL100M, and the soya lecithin may be of any model number, for example it may be soya lecithin S75 or soya lecithin S100.

In some embodiments of the present disclosure, the nose-to-brain delivery formulation of nimodipine contains 5-40 parts by weight nimodipine, 40-200 parts by weight diethylene glycol monoethyl ether, 40-160 parts by weight phospholipid, 700-850 parts by weight propylene glycol dicaprylate/dicaprate, preferably, the phospholipid can be lecithin or soya lecithin, wherein the lecithin may be of any model number, for example it may be lecithin E80 or lecithin PL100M, and the soya lecithin may be of any model number, for example it may be soya lecithin S75 or soya lecithin S100.

In certain specific embodiments of the present disclosure, the nose-to-brain delivery formulation of nimodipine comprises 15 parts by weight of nimodipine, 100 parts by weight of diethylene glycol monoethyl ether, 100 parts by weight of lecithin, 785 parts by weight of propylene glycol dicaprylate/dicaprate, wherein the model number of the lecithin may be any model number, for example, it may be lecithin E80 or lecithin PL100M.

In certain specific embodiments of the present disclosure, the nose-to-brain delivery formulation of nimodipine comprises 5-40 parts by weight of nimodipine, 40-200 parts by weight of diethylene glycol monoethyl ether, 40-160 parts by weight of phospholipids, 700-850 parts by weight of ethyl oleate, and preferably the phospholipids are lecithin or soybean lecithin, wherein the model number of the lecithin may be any model number, for example, it may be lecithin E80 or lecithin PL100M, and the model number of the soya lecithin may be any model number, for example, it may be soya lecithin S75 or soya lecithin S100.

In certain specific embodiments of the present disclosure, the nose-to-brain delivery formulation of nimodipine comprises 5 parts by weight of nimodipine, 100 parts by weight of diethylene glycol monoethyl ether, 100 parts by weight of lecithin, 795 parts by weight of ethyl oleate, wherein the model number of the lecithin may be any model number, for example, it may be lecithin E80 or lecithin PL100M.

In certain specific embodiments of the present disclosure, the nose-to-brain delivery formulation of nimodipine comprises 15 parts by weight of nimodipine, 60 parts by weight of diethylene glycol monoethyl ether, 100 parts by weight of lecithin, 825 parts by weight of ethyl oleate, wherein the model number of the lecithin may be any model number, for example, it may be lecithin E80 or lecithin PL100M.

In certain specific embodiments of the present disclosure, the nose-to-brain delivery formulation of nimodipine comprises 15 parts by weight of nimodipine, 120 parts by weight of diethylene glycol monoethyl ether, 100 parts by weight of lecithin, 765 parts by weight of ethyl oleate, wherein the model number of the lecithin may be any model number, for example, it may be lecithin E80 or lecithin PL100M.

In certain specific embodiments of the present disclosure, the nose-to-brain delivery formulation of nimodipine comprises 15 parts by weight of nimodipine, 100 parts by weight of diethylene glycol monoethyl ether, 100 parts by weight of phospholipids, 785 parts by weight of ethyl oleate, wherein the phospholipids are lecithin or soya lecithin, wherein the model number of the lecithin may be any model number, for example, it may be lecithin E80 or lecithin PL100M, and the model number of the soya lecithin may be any model number, for example, it may be soya lecithin S75 or soya lecithin S100.

In certain specific embodiments of the present disclosure, the nose-to-brain delivery formulation of nimodipine comprises 15 parts by weight of nimodipine, 100 parts by weight of diethylene glycol monoethyl ether, 60 parts by weight of phospholipids, 825 parts by weight of ethyl oleate, wherein the phospholipids are lecithin or soya lecithin, wherein the model number of the lecithin may be any model number, for example, it may be lecithin E80 or lecithin PL100M, and the model number of the soya lecithin may be any model number, for example, it may be soya lecithin S75 or soya lecithin S100.

In certain specific embodiments of the present disclosure, the nose-to-brain delivery formulation of nimodipine comprises 15 parts by weight of nimodipine, 100 parts by weight of diethylene glycol monoethyl ether, 120 parts by weight of phospholipids, 765 parts by weight of ethyl oleate, wherein the phospholipids are lecithin or soya lecithin, wherein the model number of the lecithin may be any model number, for example, it may be lecithin E80 or lecithin PL100M, and the model number of the soya lecithin may be any model number, for example, it may be soya lecithin S75 or soya lecithin $100.

In certain specific embodiments of the present disclosure, the nose-to-brain delivery formulation of nimodipine comprises the nimodipine, the solvent, the diluent and the absorption enhancer; optionally further comprises aromatizer, preferably the aromatizer is grape essence; preferably, the grape essence content is 0-50 parts by weight, more preferably is 1-10 parts by weight.

In certain specific embodiments of the present disclosure, the nose-to-brain delivery formulation of nimodipine in olfactory region and high concentration in brain tissue can acts as drug reservoirs, and the nimodipine maintains a smooth concentration in the cerebrospinal fluid for a long period of time and thus exerts its effects for a longer period of time.

In some embodiments, the nose-to-brain delivery formulation in the present disclosure is consisted of the above nimodipine, the above solvent, the above diluent and the above absorption enhancer.

In some embodiments, the nose-to-brain delivery formulation in the present disclosure is administrated through the Intelligent Integrated Nasal Drug Delivery System for Small Animals (RA-IDDS®) which is discovered by the company of the applicant, and which is disclosed in CN202021777601.0 and CN202010148279.3. The system can be used to deliver drugs to the upper nasal tract of small animals, such as mice, at a constant rate under stable anesthesia, ensuring parallelism in preclinical studies. The rodent nasal cavity is rich in olfactory nerves, which in primates are divided into olfactory and respiratory regions, and when applied to clinical trials in humans, more accurate drug delivery to the upper nasal tract is required.

In some embodiments, the nose-to-brain delivery formulation in the present disclosure has a direct transporter percentage (DTP) of cerebrospinal fluid and olfactory bulb of about 90 percent.

According to the second aspect of the present disclosure, the present disclosure provides a method for preparing the nose-to-brain delivery formulation of nimodipine, comprising: adding the nimodipine and the solvent into a glass-bottle, preferably by sonication to dissolve the nimodipine; and adding the absorption enhancer, preferably by shaking and sonication to dissolve the absorption enhancer; and, optionally, adding an aromatizer, and finally, using a diluent to the total mass of each formulation.

According to the third aspect of the present disclosure, the present disclosure provides the nose-to-brain delivery formulation of nimodipine obtained by the above method of preparing the nose-to-brain delivery formulation of nimodipine.

According to the forth aspect of the present disclosure, the present disclosure provides a method of preventing or treating ischemic disorder in a patient, in particular cerebral ischemic disorders, cerebral vasospasm caused by subarachnoid hemorrhage, and sudden deafness, comprising the step of administering a therapeutically effective amount of the nose-to-brain delivery formulation of nimodipine to a patient in need thereof.

According to the fifth aspect of the present disclosure, the present disclosure provides a use of the nose-to-brain delivery formulation of nimodipine in treating ischemic disorder by means of transnasal entry into the brain, in particular the use of treating cerebral ischemic disorders, cerebral vasospasm caused by subarachnoid hemorrhage, and sudden deafness. Especially, the present disclosure also provides a use of the above mentioned nose-to-brain delivery formulation of nimodipine in preparing for treating ischemic disorder by means of transnasal entry into the brain, in particular the use of treating cerebral ischemic disorders, cerebral vasospasm caused by subarachnoid hemorrhage, and sudden deafness.

According to the sixth aspect of the present disclosure, the present disclosure provides a composition, comprising the nose-to-brain delivery formulation of nimodipine and any suitable pharmaceutically acceptable excipient.

The term “pharmaceutically acceptable excipient” is refers to media generally accepted in the art for the delivery of biologically active agents to mammals, such as humans. These excipients are typically formulated according to a number of factors within the competence of one of ordinary skill in the art. These include, but are not limited to, the type and nature of the active agent being formulated; the subject to whom the active agent-containing composition is to be administered; the intended route of administration of the composition; and the therapeutic indication being targeted. Pharmaceutically acceptable excipients include aqueous and non-aqueous liquid media, as well as various solid and semi-solid dosage forms. Descriptions of suitable pharmaceutically acceptable excipients and the factors involved in their selection may be found in a variety of readily available sources, e.g., Remington's Pharmaceutical Sciences, 17th Edition, Mack Publishing Company, Easton, Pa. 1985, the contents of which are incorporated by application into this article.

According to the seventh aspect of the present disclosure, the present disclosure provides a use of the combination of a solvent, a diluent and an absorption enhancer in preparing of nose-to-brain delivery formulation of nimodipine, wherein the solvent is diethylene glycol monoethyl ether, and the absorption enhancer is one or more substances selected from phospholipids, dodecyl-β-maltoside, cyclopentadecanolide, polyoxyl 15 hydroxystearate, and oleoyl polyoxyl-6 glycerides.

In some embodiments, the diluent can be one or more substances selected from ethyl oleate, isopropyl myristate, medium chain triglycerides, castor oil, soyabean oil, sesame oil, oleic acid, vitamin E, glyceryl monolinoleate, propylene glycol laurate, or propylene glycol dicaprylate/dicaprate.

On the basis of common knowledge in the field, each of the above preferred conditions can be arbitrarily combined to obtain each of the preferred embodiments of the present disclosure.

The advantages of the present disclosure are as follows:
The nose-to-brain delivery formulation of nimodipine in the present disclosure, by selecting a suitable diluent, a solvent, an absorption enhancer, and a reasonable ratio thereof,
1. Increase the solubility of Nimodipine from 0.002 mg/mL to 13.5 mg/mL, more than 6000 times;
2. No nasal irritation and cilia toxicity, will not damage the nasal mucosa, high safety;
3. Avoiding the phenomenon of abnormally high brain intake caused by the destruction of nasal mucosa due to nasal irritation leading to the direct entry of drugs into the brain;
4. improving the ability of transmembrane absorption of nimodipine, the permeability coefficient can reach 2-10*10-7 (cm/s), the higher the permeability coefficient, the easier it is to pass through the mucous membrane of the nasal olfactory region and reach the olfactory bulb and the brain;
5. The nose-to-brain delivery formulation of nimodipine in this disclosure, through reasonable compounding, especially through reasonable selection of diluents with slow release effect, is conducive to the slow and smooth release of the nimodipine drug in the brain, which can form a drug reservoir and exert the drug effect for a long time;
6. The nose-to-brain delivery formulation of nimodipine of the present disclosure can directly bypass the blood-brain barrier and directly enter the brain to exert the drug effect, and the DTP (direct transfer percentage) of the cerebrospinal fluid and the olfactory bulb is close to 90%.

Therefore, the nose-to-brain delivery formulation of nimodipine in the present disclosure, does not need to pass through the blood-brain barrier to directly enter the cerebrospinal fluid and the olfactory region, and its DTP (direct transfer percentage) of the cerebrospinal fluid and the olfactory bulb is close to 90%; and the high concentration of nimodipine in the olfactory region and the brain can serve as a reservoir for the drug, and the drug can be maintained at a smoother concentration for a long period of time in the cerebrospinal fluid so as to exert a longer period of time of its medicinal efficacy. At the same time, the presently disclosed formulation improves the solubility of nimodipine through reasonable dosage compounding, and has a higher permeability coefficient, so that the drug can reach the brain tissues at a higher concentration, and can maintain a smoother concentration for a longer period of time in the brain so as to exert the efficacy for a longer period of time. The nasal brain release preparation of this disclosure is non-irritating to the nasal cavity, non-toxic to the nasal cilia, improves the patient's compliance at the same time, but also avoids the phenomenon of abnormally high brain content due to the destruction of the nasal mucosa of the preparation leading to the direct entry of the drug into the brain, which provides a new and effective way of treating neurological disorders, and has a good prospect for industrial application.

### EMBODIMENT

The method of determination of the present disclosure will be described in further detail below in connection with specific embodiments. It should be understood that the following embodiments are only exemplary to illustrate and explain the present disclosure, and should not be construed as a limitation of the scope of protection of the present disclosure. Any technology realized on the basis of the above contents of the present disclosure is covered within the scope of the present disclosure intended to be protected.

### Term Definition

In the present disclosure,

Transcutol P is diethylene glycol monoethyl ether,

Labrafil®M1944CS is oleoyl polyoxyl-6 glycerides,

HS15 is polyoxyl 15 hydroxystearate,

E80 is lecithin E80, and is a model number of lecithin,

PL100M lecithin PL100M, and is a model number of lecithin,

S75 is soya lecithin S75, and is a model number of soya lecithin,

S100 is soya lecithin S100, and is a model number of soya lecithin,

DDM is n-dodecyl-β-D-maltoside,

CMC-Na is sodium carboxymethylcellulose.

Nose-to-brain delivery: Drugs are delivered directly or indirectly into cerebrospinal fluid and brain tissue through a specific upper nasal tract, and diffuse to exert their effects in the brain.

Unless otherwise noted, the raw materials and reagents used in the following embodiments are commercially available or can be prepared by known methods.

In the present disclosure, the permeability coefficient is determined by a method known to those skilled in the art: a Franz diffusion cell is used to simulate the process of drug absorption via the nasal mucosa. The drug solution is diffused through the porcine nasal mucosa via the supply pool to the receiving pool, and the drug solution in the receiving pool is collected at different time points, the concentration is measured, and the apparent permeability coefficient of the drug absorbed via the nasal mucosa is calculated.

In the present disclosure, nasal local irritation testing was performed using a model of ciliatoxicity, which is well known to those skilled in the art: the toad palatal cilium is widely used as a commonly used model to simulate human nasal cilia [1]. The surface of the mucosa was washed and separated from the palatal mucosa 60 min after dropping the test drug on the mucosal surface, and the sustained movement of the mucosal cilia was observed under a light microscope, and its effect on the cilia was analyzed by using saline as a control [2], the cilia movement was observed, and the time required from the beginning of drug administration to the complete cessation of cilia movement was recorded, i.e., the cilium sustained movement time. The relative percentage of ciliary sustained movement time was obtained by dividing the ciliary sustained movement time of the palatal mucosa of the toads in the drug administration group by the ciliary sustained movement time of the palatal mucosa of the toads in the negative control group, and the higher the percentage, the lower the toxicity to the cilium of the preparation, and the higher the safety of the preparation. The higher the percentage, the less toxic the formulation is to the cilia, and the higher the safety of the formulation. The relative percentage of ciliary sustained motion time is greater than 85%, which is the threshold for the safety of the formulation. ([1] Jiang XG, Cui JB. Nasal mucosal ciliotoxicity of drugs and evaluation methods [J]. Journal of Pharmacy, 1995, 30:848-53. [2] Li XF, Li XG, Ma ZQ, et al. Preparation and safety study of menthol nasal in situ gel [J]. Journal of Pharmacy Practice, 2017, 3 (4): 321-4.)

It should be understood by those skilled in the art that in order to increase the compliance of the subject to the nasal brain delivery formulation, aromatizer such as grape essence may also be added to adjust the taste of the nasal brain delivery formulation, and accordingly, the amount of diluent is reduced accordingly with the addition of aromatizer, e.g., the addition of 2.5 parts by weight of grape essence with a corresponding reduction of 2.5 parts by weight of diluent, this does not constitute a Limitations.

### Embodiment 1

The formulated amount of each formulation, as shown in Table 1, was prepared at room temperature in the following manner, i.e., weighing the formulated amount of nimodipine and the corresponding solvent into a glass bottle, sonication to make the solution; and then according to the specific components of each formulation, adding the corresponding amount of absorption enhancer, vibration and sonication to make the solution; and finally, adding the formulated amount of diluent to the total mass of the formulations to reach 1 g, rotating and mixing for 30 minutes, so that the dispersion of homogeneous to obtain the following formulations; and testing the permeability coefficient.

TABLE 1

| Formulation number | | Embodiment 1-1 | Embodiment 1-2 |
|---|---|---|---|
| Nimodipine (mg) | | 15 | 15 |
| solvent | Transcutol P | 100 | 100 |
| | (mg) | | |
| Absorption | DDM | \ | 2 |
| enhancer | (mg) | | |

| | | |
|---|---|---|
| castor oil (mg) | 885 | 883 |
| permeability coefficient | 2.21 ± 0.16 | 2.26 ± 0.45 |
| (cm/s) 10⁻⁷ | | |

Conclusion: The permeability coefficients of nimodipine nose-to-brain delivery formulations, using Transcutol P as a solvent and castor oil as a diluent, were all above 2; the addition of DDM had little effect on the permeability coefficients.

### Embodiment 2

The formulated amount of each formulation, as shown in Table 2, was prepared in accordance with the method described in embodiment 1, weighing the formulated amount of nimodipine and the corresponding solvent into a glass bottle, sonication to make the solution; and then according to the specific components of each formulation, adding the corresponding amount of absorption enhancer, vibration and sonication to make the solution; and finally, adding the formulated amount of diluent to the total mass of the formulations to reach 1 g, rotating and mixing for 30 minutes, so that the dispersion of homogeneous to obtain the following formulations; and testing the permeability coefficient.

TABLE 2

| | | Formulation number | | | | | |
|---|---|---|---|---|---|---|---|
| | | Embodiment | Embodiment | Embodiment | Embodiment | Embodiment | Embodiment |
| | | 2-1 | 2-2 | 2-3 | 2-4 | 2-5 | 2-6 |
| Nimodipine (mg) | | 15 | 15 | 15 | 15 | 15 | 15 |
| Transcutol P (mg) | | 100 | 100 | 100 | 100 | 100 | 100 |
| solvent | species | cyclopenta- | cyclopenta- | cyclopenta- | lecithin | lecithin | HS15 |
| | | decanolide | decanolide | decanolide | | | |
| | dosage | 40 | 40 | 40 | 40 | 40 | 1 |
| | (mg) | | | | | | |
| diluent | species | castor oil | isopropyl | oleic acid | oleic acid | isopropyl | oleic acid |
| | | | myristate | | | myristate | |
| | dosage | 845 | 845 | 845 | 845 | 845 | 884 |
| | (mg) | | | | | | |

| | | | | | | |
|---|---|---|---|---|---|---|
| permeability | 2.38 ± 1.12 | 3.03 ± 0.96 | 1.99 ± 0.48 | 3.34 ± 0.85 | 3.99 ± 1.20 | 2.54 ± 0.44 |
| coefficient | | | | | | |
| (cm/s) 10⁻⁷ | | | | | | |

Conclusion: The choice of lecithin and cyclopentadecanolide as absorption enhancers helped to increase the permeability coefficient of the nasal mucosa; isopropyl myristate samples were the best absorbed among the different diluents; the permeability coefficients were at critical values when 4% cyclopentadecanolide was chosen as the absorption enhancer and oleic acid was chosen as the diluent.

### Embodiment 3

This embodiment compares different weight portions of lecithin, cyclopentadecanolide, lecithin plus cyclopentadecanolide, and lecithin plus HS15 as absorption enhancer on the permeability coefficients of the nasal mucosa of the nilmodipine nose-to-brain delivery formulation of the present disclosure.

The formulated amount of each formulation, as shown in Table 3 and 4, was prepared at room temperature in the following manner, i.e., weighing the formulated amount of nimodipine and the corresponding solvent into a glass bottle, sonication to make the solution; and then according to the specific components of each formulation, adding the corresponding amount of absorption enhancer, vibration and sonication to make the solution; and finally, adding the formulated amount of diluent to the total mass of the formulations to reach 1 g, rotating and mixing for 30 minutes, so that the dispersion of homogeneous to obtain the following formulations; and testing the permeability coefficient.

TABLE 3

| Formulation number | | Formulation 3-1 | Formulation 3-2 | Formulation 3-3 | Formulation 3-4 |
|---|---|---|---|---|---|
| nimodipine (mg) | | 15 | 15 | 15 | 15 |
| solvent | Transcutol P | 100 | 100 | 100 | 100 |
| | (mg) | | | | |
| Formulation | species | lecithin | | cyclopentadecanolide | |
| | Dosage | 100 | 160 | 100 | 160 |
| | (mg) | | | | |
| diluent | isopropyl | 785 | 725 | 785 | 725 |
| | myristate | | | | |
| | (mg) | | | | |

| | | | | |
|---|---|---|---|---|
| permeability coefficient | 5.02 ± 0.94 | 3.18 ± 1.14 | 3.87 ± 0.75 | 3.06 ± 0.86 |
| (cm/s) 10⁻⁷ | | | | |

TABLE 4

| Formulation number | | Formulation 3-5 | Formulation 3-6 | Formulation 3-7 | Formulation 3-8 |
|---|---|---|---|---|---|
| nimodipine (mg) | | 15 | 15 | 15 | 15 |
| Good | Transcutol P | 100 | 100 | 100 | 100 |
| solven | (mg) | | | | |
| Formulation | species | Lecithin plus cyclopentadecanolide | | Lecithin plus HS15 | |
| | dosage (mg) | 100 + 40 | 100 + 100 | 100 + 1 | 100 + 2.5 |

| | | | | |
|---|---|---|---|---|
| isopropyl myristate | 745 | 685 | 784 | 782.5 |
| (mg) | | | | |
| permeability coefficient | 3.32 ± 1.08 | 2.57 ± 0.75 | 4.30 ± 0.89 | 4.17 ± 0.60 |
| (cm/s) 10−7 | | | | |

Conclusions: Firstly, for the amount of absorption enhancer, it was found that the highest values of the corresponding porcine nasal mucosal permeability coefficients were obtained when 10% lecithin or cyclopentadecanolide was used (see Formulation 3-1 and Formulation 3-2; Formulation 3-3 and Formulation 3-4 in Table 3, respectively). Secondly, for the species of absorption enhancer, it was found that the highest values of the corresponding porcine nasal mucosal permeability coefficients were obtained when 10% lecithin was used (see Formulation 3-1 in Table 3). However, it was found that the use of lecithin in combination with both cyclopentadecanolide and HS15 for the absorption enhancers led to a decrease in the permeability coefficients (see Formulations 3-5 to 3-8 in Table 4, respectively), and that the permeability coefficients decreased with an increase in the amount of cyclopentadecanolide and HS15 (see Formulations 3-5 to 3-8 in Table 4).

Accordingly, using phospholipids alone as absorption enhancers, the nimodipine nose-to-brain delivery formulation of this embodiment has an optimal permeability coefficient. The amount of phospholipid can be selected as 20-200 parts by weight, preferably 40-160 parts by weight.

### Embodiments 4

This embodiments compares the effects of using lecithin E80, lecithin PL100M, soya lecithin S75 and soya lecithin S100 as absorption enhancers and the use of isopropyl myristate, propylene glycol dicaprylate/dicaprate, ethyl oleate, and propylene glycol laurate as diluents on the appearance, nasal local irritation, and nasal mucosal permeability coefficients of the nose-to-brain delivery formulation of nimodipine of the present disclosure.

The formulations shown in Tables 5 and 6 below were prepared in a method similar to that of embodiment 1 by weighing the formulated amount of nimodipine 15 mg and the solvent Transcutol P 100 mg into a glass vial, ultrasonically dissolving them (for about 7 minutes), adding the phospholipids corresponding to the different formulations, and shaking and ultrasonically dissolving them (for about 5 minutes); and finally, adding the diluents corresponding to the different formulations until the total mass was 1 g, rotating and mixing for 30 minutes, and then mixing for 30 minutes. Finally, add the diluents corresponding to different formulations to a total mass of 1 g, rotate and mix for 30 minutes to make a uniform dispersion; the following formulations were obtained.

TABLE 5

| | | Formulation number | | |
|---|---|---|---|---|
| | | Formula- | Formula- | Formula- |
| | | tion 4-1 | tion 4-2 | tion 4-3 |
| Nimodipine (mg) | | 15 | 15 | 15 |
| solvent | Transcutol P | 100 | 100 | 100 |
| | (mg) | | | |
| Absorption | species | E80 | PL100M | S75 |
| enhancer:phos- | dosage | 100 | 100 | 100 |
| pholipids | (mg) | | | |
| Diluent | species | isopropyl myristate | | |
| | dosage | 785 | 785 | 785 |
| | (mg) | | | |

| Appearance | Light yellow | yellow | Orange |
|---|---|---|---|
| (room temperature) | transparent | transparent | transparent |
| permeability coefficient | 5.6 ± 0.5 | 8.5 ± 1.4 | 7.0 ± 2.2 |
| (cm/s) 10⁻⁷ | | | |
| nasal local irritation | Reach the | Reach the | Reach the |
| | standard | standard | standard |

TABLE 6

| | | Formulation number | | |
|---|---|---|---|---|
| | | Formula- | Formula- | Formula- |
| | | tion 4-4 | tion 4-5 | tion 4-6 |
| Nimodipine (mg) | | 15 | 15 | 15 |
| solvent | Transcutol | 100 | 100 | 100 |
| | P (mg) | | | |
| Absorption | species | PL100M | | |
| enhancer:phos- | dosage | 100 | 100 | 100 |
| pholipids | (mg) | | | |
| Diluent | species | ethyl oleate | propylene | propylene |
| | | | glycol | glycol |
| | | | laurate | dicaprylate/ |
| | | | | dicaprate |
| | dosage | 785 | 785 | 785 |
| | (mg) | | | |

| Appearance | yellow | Orange | yellow |
|---|---|---|---|
| (room temperature) | transparent | transparent | transparent |
| permeability | 9.6 ± 3.2 | 2.5 ± 0.6 | 5.1 ± 1.3 |
| coefficient | | | |
| (cm/s) 10⁻⁷ | | | |
| nasal local irritation | Reach the | Reach the | Reach the |
| | standard | standard | standard |

Conclusion: Firstly, for phospholipids as absorption enhancers, lecithin PL-100M and soya lecithin S75 showed better transnasal mucosal absorption. Secondly, for diluents, ethyl oleate and isopropyl myristate showed the best transnasal mucosal absorption (see Table 6, Formulation 4-4 and Table 5, Formulation 4-2), followed by propylene glycol dicaprylate/dicaprate (see Table 6, Formulation 4-6).

Combining the above effects of the absorption enhancer and diluent on the appearance, nasal local irritation and nasal mucosal permeability coefficient of the presently disclosed nimodipine nose-to-brain delivery formulation of nimodipine, the presently disclosed nose-to-brain delivery formulation of nimodipine preferably employs lecithin or soya lecithin as the absorption enhancer; and preferably employs one or more of ethyl oleate, isopropyl myristate, and propylene glycol dicaprylate/dicaprate as the diluent.

### Embodiment 5

According to the formulation shown in Table 7 to prepare the nasal brain delivery formulation of nimodipine in the present disclosure, the specific preparation method is as follows: weighing the formula amount of nimodipine and solvent Transcutol P to the glass bottle, ultrasonicate to make the solution; and then, according to the specific components of each formula, add the corresponding amount of absorption enhancer (a variety of phospholipids), shake and ultrasonicate to dissolve; and finally add a formula amount of diluent ethyl oleate to the formula to the total mass of 1 g, rotary mixing for 30 minutes, so that the dispersion of the uniformity of the formulation can be obtained as follows.

TABLE 7

| | Dosage of Lecithin | | | | | | |
|---|---|---|---|---|---|---|---|
| | PL-100M | | | Dosage of Transcutol P | | | |
| | Formulation number | | | | | | |
| | Formulation | Formulation | Formulation | Formulation | Formulation | Formulation | Formulation |
| | 5-1 | 5-2 | 5-3 | 5-4 | 5-5 | 5-6 | 5-7 |
| Nimodipine | 15 | 15 | 15 | 15 | 15 | 15 | 15 |
| (mg) | | | | | | | |
| Transcutol | 100 | 100 | 100 | 60 | 80 | 120 | 140 |
| P(mg) | | | | | | | |
| Lecithin | 60 | 80 | 120 | 100 | 100 | 100 | 100 |
| PL-100M | | | | | | | |
| ethyl oleate | 825 | 805 | 765 | 825 | 805 | 765 | 745 |
| Purified | — | — | — | — | — | — | — |
| Lecithin | | | | | | | |
| Domestic | — | — | — | — | — | — | — |
| soya | | | | | | | |
| lecithin | | | | | | | |
| soya | — | — | — | — | — | — | — |
| lecithin | | | | | | | |
| S75 | | | | | | | |
| soya | — | — | — | — | — | — | — |
| lecithin | | | | | | | |
| S100 | | | | | | | |
| density | 0.8992 | 0.9007 | 0.9040 | 0.8990 | 0.8898 | 0.8911 | 0.9052 |
| (g/ml) | | | | | | | |
| permeability | 4.8 ± 3.3 | 4.7 ± 2.9 | 5.5 ± 1.2 | 6.2 ± 1.8 | 14.5 ± 3.2 | 19.3 ± 2.8 | 6.8 ± 0.8 |
| coefficient | | | | | | | |
| (cm/s) 10⁻⁷ | | | | | | | |

| | Lower | | | | |
|---|---|---|---|---|---|
| | concen- | | | | |
| | tration | soya lecithin | | | |
| | Formulation number | | | | |
| | Formulation | Formulation | Formulation | Formulation | Formulation |
| | 5-8 | 5-9 | 5-10 | 5-11 | 5-12 |
| Nimodipine | 5 | 15 | 15 | 15 | 15 |
| (mg) | | | | | |
| Transcutol | 100 | 100 | 100 | 100 | 100 |
| P(mg) | | | | | |
| Lecithin | 100 | — | — | — | — |
| PL-100M | | | | | |
| ethyl oleate | 795 | 785 | 785 | 785 | 785 |
| Purified | — | 100 | — | — | — |
| Lecithin | | | | | |
| Domestic | — | — | 100 | — | — |
| soya | | | | | |
| lecithin | | | | | |
| soya | — | — | — | 100 | — |
| lecithin | | | | | |
| S75 | | | | | |
| soya | — | — | — | — | 100 |
| lecithin | | | | | |
| S100 | | | | | |
| density | 0.8991 | 0.9016 | 0.9064 | 0.9019 | 0.9041 |
| (g/ml) | | | | | |
| permeability | 14.9 ± 2.0 | 7.5 ± 0.9 | 6.8 ± 1.0 | 5.7 ± 2.0 | 8.7 ± 3.6 |
| coefficient | | | | | |
| (cm/s) 10⁻⁷ | | | | | |

As can be seen from Table 7 above, firstly, the type of phospholipid has little effect on the permeability coefficient of the present disclosed nimodipine nose-to-brain delivery formulation (see Table 7). Secondly, Transcutol P and phospholipids in the range of 60-140 parts by weight are still able to meet the requirements of the permeability coefficient and nasal local irritation for nose-to-brain delivery formulations (see Table 7). Finally, a decrease in API concentration reduces local absorption, and a high API concentration favors local absorption (see Table 7 Formulations 5-8).

### Comparative Embodiment 1

The Comparative formulations shown in Tables 8 and 9, weigh the formulated amount of nimodipine and the corresponding solvent into a glass bottle, ultrasonicate to dissolve; then according to the specific components of each formulation, add the corresponding amount of absorption enhancer and/or surfactant, shake and ultrasonicate to dissolve; and finally, add the formulated amount of diluent to the total mass of the formulations to reach 1 g, rotate and mix for 30 minutes to make a homogeneous dispersion.

TABLE 8

| | | Formulation number | | | |
|---|---|---|---|---|---|
| | | Compar- | Compar- | Compar- | Compar- |
| | | ative | ative | ative | ative |
| | | Embodi- | Embodi- | Embodi- | Embodi- |
| | | ment 1-1 | ment 1-2 | ment 1-3 | ment 1-4 |
| nimodipine (mg) | | 25 | 25 | 5 | 5 |
| Good solent | benzyl | 9 | 9 | / | / |
| (mg) | alcohol | | | | |
| | PEG400 | 100 | 100 | / | / |
| | ethanol | 50 | 50 | 30 | 30 |
| | Transcutol | / | / | 45 | 45 |
| | P | | | | |
| surfactant | species | Tween 80 | Tween 20 | Tween 80 | Labrafil ® |
| | | | | | M1944cs |
| | Dosage | 5 | 25 | 1 | 40 |
| | (mg) | | | | |
| Absorption | DDM | 0.43, 2.1 | 0.43, 2.1 | 2.1 | / |
| enhancer | (mg) | | | | |

| | | | | |
|---|---|---|---|---|
| castor oil (mg) | 810.57 | 790.57 | 916.9 | 884 |

TABLE 9

Formulation number

| | | | Comparative | Comparative |
|---|---|---|---|---|
| | | | Embodiment | Embodiment |
| | | | 1-5 | 1-6 |
| Nimodipine (mg) | | | 25 | 15 |
| | solvent (mg) | benzyl | 9 | 5.4 |
| | | alcohol | | |
| | | PEG400 | 100 | 60 |
| | | ethanol | 50 | 50 |
| | | Transcutol P | / | / |
| | surfactant | species | Tween 20 | Tween 20 |
| | | Dosage (mg) | 2.5 | 1.5 |
| | Absorption | DDM (mg) | 2.1 | 2.1 |
| | enhancer | | | |

| | | |
|---|---|---|
| castor oil (mg) | 811.4 | 866 |

The results of the experiment are shown in Table 10 and Table 11 below:

TABLE 10

| | Formulation number | | | | | |
|---|---|---|---|---|---|---|
| | Comparative | | Comparative | | Comparative | Comparative |
| | Embodiment 1-1 | | Embodiment 1-2 | | Embodiment 1-3 | Embodiment 1-4 |
| Nasal local | reach the | | reach the | | reach the | reach the |
| irritation | standard | | standard | | standard | standard |
| permeability | 0.04% DDM | 0.2% DDM | 0.04% DDM | 0.2% DDM | 0.39 ± 0.14 | 0.46 ± 0.22 |
| coefficient | 0.64 ± 0.19 | 0.97 ± 0.39 | 0.54 ± 0.21 | 1.24 ± 0.40 | | |
| (cm/s) 10⁻⁷ | | | | | | |

TABLE 11

Formulation number

| | Comparative | Comparative |
|---|---|---|
| | Embodiment | Embodiment |
| | 1-5 | 1-6 |
| Nasal local | reach the | reach the |
| irritation | standard | standard |
| permeability | 0.95 ± 0.19 | 1.28 ± 0.42 |
| coefficient | | |
| (cm/s) 10⁻⁷ | | |

### Conclusion:

When alcohols are used as solvents in the nose-to-brain delivery formulation, the permeability coefficients of nimodipine nose-to-brain delivery formulations, even with the addition of surfactants or/and absorption enhancers, remain low and do not meet the requirements for nose-to-brain release formulations that enter the brain via the mucous membranes of the olfactory region of the nose.

### Comparative Embodiment 2

The formulations shown in Tables 12 below, were prepared in accordance with the conventional methods of those skilled in the art and may also be prepared in accordance with the analogous method of Comparative Embodiment 1;

Tabel 12

| | | Formulation number | | | | |
|---|---|---|---|---|---|---|
| | | Comparative | Comparative | Comparative | Comparative | Comparative |
| | | Embodiment 2-1 | Embodiment 2-2 | Embodiment 2-3 | Embodiment 2-4 | Embodiment 2-5 |
| nimodipine (mg) | | 15 | 15 | 15 | 15 | 15 |
| Transcutol P (mg) | | 100 | 100 | 100 | 100 | 100 |
| Absorption | species | polyoxyethylene | Tween 20 | PL100M | PL100M | PL100M |
| enhancer | | castor oil ELP | | | | |
| | dosage | 40 | 20 | 100 | 100 | 100 |
| | (mg) | | | | | |
| diluent | spices | castor oil | oleic acid | propylene | glyceryl | propylene |
| | | | | glycol | mono and | glycol |
| | | | | monocaprylate | dicaprylocaprate | monolaurate |
| | dosage | 845 | 865 | 785 | 785 | 785 |
| | (mg) | | | | | |

| | | | | | |
|---|---|---|---|---|---|
| permeability | 1.27 ± 1.10 | 1.74 ± 0.30 | 1.6 ± 0.1 | coagulation | coagulation |
| coefficient | | | | occurs, | occurs, |
| (cm/s) 10⁻⁷ | | | | permeability | permeability |

| | |
|---|---|
| coefficient | coefficient |
| Unmeasured | Unmeasured |

Conclusion : When using polyoxyethylene castor oil ELP or Tween 80 as absorption enhancer in the preparation of nose-to-brain delivery of nimodipine, the permeability coefficient is difficult to meet the requirements of nasal brain delivery; when using lecithin as the absorption enhancer, the type of diluent will affect the permeability coefficient, for example, using propylene glycol monocaprylate as diluent, the permeability coefficient is only 1.6*10⁻⁷ cm/s; when using glyceryl mono and dicaprylocaprate or propylene glycol monolaurate, coagulation occurs, which does not meet the basic requirements of nasoencephalic delivery formulations.

Pharmacologic Embodiment 1: Comparison with Intravenous Administration of a Control Formulation

Pharmacokinetic data was assayed using Formulations 5-8 in embodiment 5. The nose-to-brain delivery formulation of nimodipine of Formulations 5-8 was placed in a transnasal drug delivery device (RA-IDDS® Intelligent Integrated Nasal Drug Delivery System for Small Animals developed by the present applicant company, as disclosed in CN202021777601.0 and CN202010148279.3), and the prescribed dose of the drug was applied to the mice transnasally, and the nasal delivery was made in a state of gas anesthesia for 2 min. The pharmacokinetics and distribution of the formulation administered via the nose were evaluated at 2, 5, 15, 30, 60, 120, 180 min after the administration of the drug, and plasma, olfactory bulb, and cortical brain tissue were collected by sampling methods commonly used by those skilled in the art, for the collection of cerebrospinal fluid, the method of evacuating the blood and then collecting the cerebrospinal fluid was adopted, which can try to avoid cerebrospinal fluid contamination by the blood, and make the test results more accurate.

In this embodiment, ST-0 refers to Nimotop® injection (size 50 ml/10 mg) manufactured by Bayer, and ST202-Y refers to formulations 5-8 prepared in embodiment 5; the DMPK comparison data for nasal administration of nose-to-brain delivery formulation versus intravenous administration of Nimotop® injection are shown in Table 13 below:

TABLE 13

| | | plasma | | | |
|---|---|---|---|---|---|
| | dosage | Cₘₐₓ | Tₘₐₓ | AUC₀₋ₜ | t_{1/2} |
| administration | mg/kg | ng/ml | min | min · ng/ml | min |
| Intravenous | 2 | 596.0 | 2 | 14302 | 49 |
| Control ST-0 | | | | | |
| Nose-to-brain | | 33.8 | 15 | 3753 | 307 |
| delivery ST202-Y | | | | | |

| | | cerebrospinal fluid (CSF) | | | | | |
|---|---|---|---|---|---|---|---|
| | dosage | Cₘₐₓ | CSF/ | Tₘₐₓ | AUC₀₋ₜ | CSF/ | t_{1/2} |
| administration | mg/kg | ng/ml | plasma | min | min · ng/ml | plasma | min |
| Intravenous | 2 | 4.2 | 0.01 | 2 | 149 | 0.01 | 242 |
| Control ST-0 | | | | | | | |
| Nose-to-brain | | 3.8 | 0.11 | 180 | 229 | 0.06 | / |
| delivery ST202-Y | | | | | | | |
| | | olfactory bulb (OB) | | | | | |
| | dosage | Cₘₐₓ | OB/ | Tₘₐₓ | AUC₀₋ₜ | OB/ | t_{1/2} |
| administration | mg/kg | ng/ml | plasma | min | min · ng/ml | plasma | min |
| Intravenous | 2 | 1547.4 | 2.60 | 2 | 29834 | 2.09 | 24 |
| Control ST-0 | | | | | | | |
| Nose-to-brain | | 1322.6 | 39.13 | 5 | 55257 | 14.72 | 43 |
| delivery ST202-Y | | | | | | | |
| | | cortical brain tissue | | | | | |
| | | | cortical | | | cortical | |
| | | | brain | | | brain | |
| | dosage | Cₘₐₓ | tissue/ | Tₘₐₓ | AUC₀₋ₜ | tissue/ | t_{1/2} |
| administration | mg/kg | ng/ml | plasma | min | min · ng/ml | plasma | min |
| Intravenous | 2 | 1744.4 | 2.93 | 2 | 29435 | 2.06 | 36 |
| Control ST-0 | | | | | | | |
| Nose-to-brain | | 158.0 | 4.67 | 5 | 10503 | 2.80 | 84 |
| delivery ST202-Y | | | | | | | |

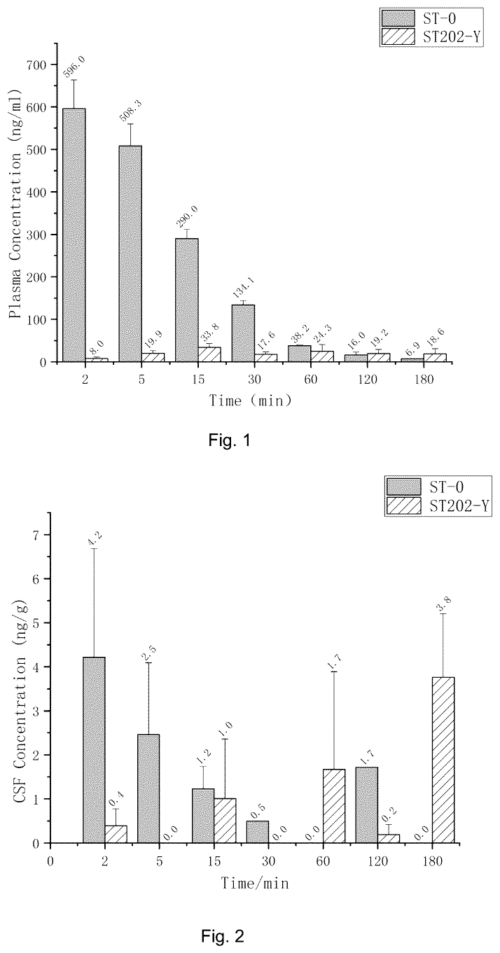

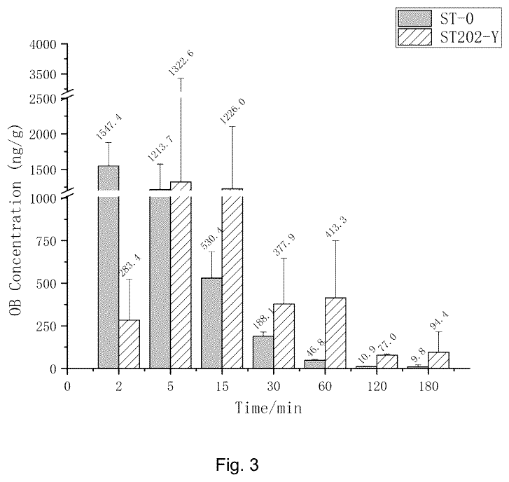

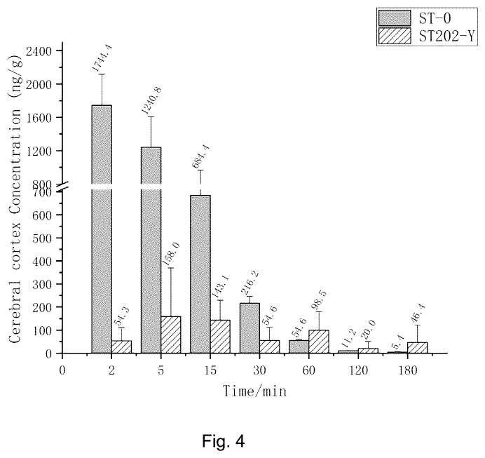

When ST-O was administered intravenously to mice, it takes 2 mins to finish the administration, with the improved formulation ST202-Y of the present disclosure administered to mice via nasal route, the blood drug concentrations in plasma, cerebrospinal fluid, olfactory bulb, and cortical brain tissues are shown in FIG. 1, FIG. 2, FIG. 3, and FIG. 4, respectively.

As can be seen from FIG. 1, the plasma drug exposure of nasal administration is only ¼ of that of intravenous injection, which can reduce peripheral adverse reactions and has a higher safety profile.

The clinical use of ST-0 Nimotop® injection is prolonged intravenous administration, which is not convenient for direct control in animal experiments, so the cerebrospinal fluid blood ratio, cerebral blood ratio, and DTP % (percentage of direct drug transfer) in the PK data are the most informative; the cerebrospinal fluid blood ratio and the cerebral blood ratio of the nasal administration are higher than that of the intravenous control administration by a factor of about 10.

The most prominent indication for nimodipine is subarachnoid hemorrhage, where the cerebrospinal fluid itself is in the subarachnoid space. As shown in FIGS. 2 and 3, the exposure of the drug in the cerebrospinal fluid of the nasal administration is more than twice as much as that of the intravenous administration in 3 hours, which is a significant advantage; the nasal brain delivery formulation of the present disclosure, after nasal administration, has a high drug concentration in the olfactory bulb, which can act as a reservoir of the drug, releasing the drug slowly into the cerebrospinal fluid for a long period of time; in 3 h, the cerebrospinal fluid content of the intravenous ST-O preparation has dropped to its lowest value, while the nasal administration of the improved nasal brain delivery formulation of the present disclosure, ST202-Y, is still at its second peak at 3 h and is still exerting its drug effect. As shown in FIG. 4, ST-0 is highly susceptible to enter the brain by stimulating blood vessels to break the blood-brain barrier, resulting in a transient increase in brain content. However, the nasal brain delivery formulation of the present disclosure is safe and non-irritating with high targeting of both cerebrospinal fluid and cortical brain tissue.

Drugs can be absorbed into the brain via two pathways after nasal administration: the systemic pathway and the olfactory pathway. In order to more clearly describe the direct nose-brain diversion, i.e. brain targeting of the nose-to-brain delivery formulations of the present application, it is expressed as a DTP % value (percentage of drug directly transported through the nose),

$$DTP \% = ( {B}_{in} - {B}_{x} ) / {B}_{in} \times 100 \% ,$$

$$\frac{{B}_{iv}}{{P}_{iv}} = \frac{{B}_{x}}{{P}_{in}}$$

Bₓ indicates the AUC value of part of the drug entering the brain through the BBB via the somatic circulation after nasal administration;

Pᵢᵥ, Bᵢᵥ, Pᵢₙ, Bᵢₙ indicate the AUC values in blood and brain after intravenous administration or nasal administration, respectively.

After calculation, DTP % in cerebrospinal fluid is 83%, DTP % in olfactory bulb is 86%; The nose-to-brain delivery formulation of nimodipine in the present application has a DTP % of 83% in the cerebrospinal fluid and 86% in the olfactory bulb, and it has a strong targeting ability as it directly enters into the targeting portion such as the cerebrospinal fluid and the brain tissue without having to pass through the blood-brain barrier. The most important indication of nimodipine is subarachnoid hemorrhage, and the cerebrospinal fluid itself is in the subarachnoid space, so the application's nimodipine nose-to-brain delivery formulation has obvious efficacy and significant advantages.

Pharmacologic Embodiment 2: Comparison with Oral Administration of a Control Formulation

ST-1 was dissolved in 0.5% CMC-Na to make a 1.5 mg/mL gavage solution and subjected to an overall absorption and distribution test, with collection of plasma, cerebrospinal fluid, and brain tissue, at time points 5, 15, 30, 60, 120, 240, 360, and 480 mins, in parallel with 6 rats; the pharmacokinetics and distribution of the orally administered drug was evaluated and compared to the nose-to-brain delivery formulations of this application.

For the purposes of this pharmacologic embodiment:
ST-1 refers to the Nimotop® tablet manufactured by Bayer;
ST202-Y-1 refers to a 5 mg/g nose-to-brain delivery formulation, using Formulations 5-8 prepared in Embodiment 5 of the present application, with a mouse dose of 1 mg/kg, and administered using the RA-IDDS® Intelligent Integrated Nasal Delivery System for Small Animals developed by the present applicant company;
ST202-Y-3 refers to a 15 mg/g nose-to-brain delivery formulation, with a mice dose of 3 mg/kg, administered using the RA-IDDS® Intelligent Integrated Nasal Drug Delivery System for Small Animals developed by the Company of the present applicant; the method of preparation of the ST202-Y-3 formulation: weigh 1.35 g of nimodipine, 10.08 g of Transcutol P to make it soluble, then add soya lecithin 9 g, stirred to dissolve, weighed 0.23 g of grape essence, added to the above solution, using ethyl oleate to the total amount of the formulation to 90 g, stirring to obtain nose-to-brain delivery formulation of nimodipine in 15 mg/g.

The experimental results are shown in Table 14 and Table 15 below;

TABLE 14

| | | plasma | | | |
|---|---|---|---|---|---|
| | Dosage | Cₘₐₓ | Tₘₐₓ | AUC₀₋ₜ | t_{1/2} |
| administration | mg/kg | ng/ml | min | min ng/ml | min |
| Oral control | 10.5 | 18.9 | 30 | 1047 | 32 |
| ST-1 | | | | | |
| Nose-to-brain | 1 | 25.9 | 15 | 1836 | 79 |
| delivery | | | | | |
| ST202-Y-1 | | | | | |
| Nose-to-brain | 3 | 103.7 | 15 | 10382 | 88 |
| delivery | | | | | |
| ST202-Y-3 | | | | | |

TABLE 15

| | | brain | | | AUC₀₋ₜ | | |
|---|---|---|---|---|---|---|---|
| | Dosage | Cₘₐₓ | brain/ | Tₘₐₓ | min | brain/ | t_{1/2} |
| administration | mg/kg | ng/ml | plasma | min | ng/ml | plasma | min |
| Oral control ST-1 | 10.5 | 33.2 | 176% | 30 | 2107 | 201% | 43 |
| Nose-to-brain | 1 | 107.0 | 413% | 15 | 7610 | 414% | 211 |
| delivery ST202-Y-1 | | | | | | | |
| Nose-to-brain | 3 | 307.1 | 296% | 15 | 28489 | 274% | 136 |
| delivery ST202-Y-3 | | | | | | | |

CONCLUSION: Compared with oral control ST-1, the nose-to-brain delivery formulation of the present disclosure has obvious advantages, reaching the peak quickly; the dosage needs only 1/10th of the oral dosage, the plasma and brain drug exposure is 1.75 and 3.61 times as much as that of the oral control ST-1, and the peak concentration of the drug in the plasma and the brain is 1.37 and 3.22 times as much as that of the oral control ST-1, so that the efficacy of the nose-to-brain delivery formulation of the present disclosure is obvious,

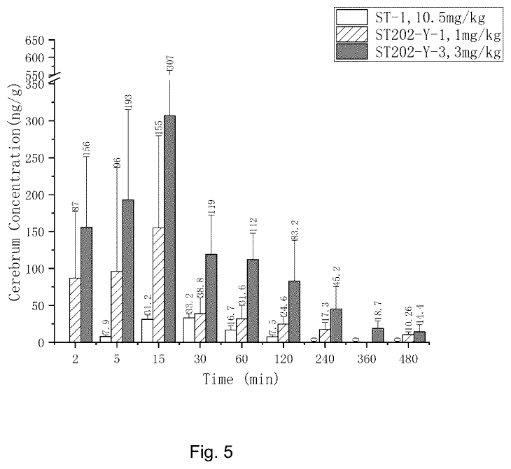

Exposure in the brain can be as high as to more than 10 times the oral dose when the dose is ⅓ of the oral dose (as shown in FIG. 5);

The nose-to-brain delivery formulation of the disclosure has a longer half-life in the plasma and brain of mice compared to the oral control ST-1, which reduces the number of administrations and enhances compliance.

### Pharmacologic Embodiment 3: Distribution of Rat Tissues

Adopting a method similar to that of Pharmacological Embodiment 2, the oral control ST-1 was formulated in the same way as Pharmacological Embodiment 2, and the nose-to-brain delivery formulation ST202-Y-4 in this Pharmacological Embodiment was prepared at a formulation concentration of 15 mg/g, which was prepared as follows: in a reactor, 0.405 kg of nimodipine and 3.20 kg of Transcutol P were added, and stirred until dissolution, then adding 2.70 kg of lecithin PL-100M, stirred to dissolve, then added 67.50 g of grape essence and 20.59 kg of ethyl oleate, stirred to dissolve to obtain the formulation ST202-Y-4; using the same drug delivery equipment as in Pharmacological embodiment 2 for the administration to rats;

The experimental results are shown in Table 16 below:

TABLE 16

| | | plasma | | | | |
|---|---|---|---|---|---|---|
| | dosage | Tₘₐₓ | Cₘₐₓ | C_{120 min} | AUC_{0-120 min} | C_{120 min}/ |
| administration | mg/kg | min | ng/ml | ng/ml | min · ng/ml | Cₘₐₓ % |
| Oral control ST-1 | 6 | 15 | 3.21 | 0.27 | 182.4 | 8.41 |
| Nose-to-brain | 0.6 | 15 | 17.15 | 4.08 | 932.4 | 23.79 |
| delivery ST202-Y-4 | | | | | | |

| | | cerebrospinal fluid (CSF) | | | | | | |
|---|---|---|---|---|---|---|---|---|
| | dosage | Tₘₐₓ | Cₘₐₓ | C_{120 min} | AUC_{0-120 min} | C_{120 min}/ | CSF/ | AUC_{0-120 min} |
| administration | mg/kg | min | ng/ml | ng/ml | min · ng/ml | Cₘₐₓ % | plasma | CSF/plasma |
| Oral control ST-1 | 6 | 15 | 1.50 | 0.11 | 34.2 | 7.33 | 0.47 | 0.19 |
| Nose-to-brain | 0.6 | 120 | 3.52 | 3.52 | 167.4 | 100.00 | 0.21 | 0.18 |
| delivery ST202-Y-4 | | | | | | | | |
| | | brain | | | | | Cₘₐₓ | AUC_{0-120 min} |
| | dosage | Tₘₐₓ | Cₘₐₓ | C_{120 min} | AUC_{0-120 min} | C_{120 min}/ | brain/ | brain/ |
| administration | mg/kg | min | ng/g | ng/g | min · ng/g | Cₘₐₓ % | plasma | plasma |
| Oral control ST-1 | 6 | 15 | 3.42 | 0.49 | 235.8 | 14.33 | 1.07 | 1.29 |
| Nose-to-brain | 0.6 | 15 | 39.47 | 8.22 | 2307.0 | 20.83 | 2.30 | 2.47 |
| delivery ST202-Y-4 | | | | | | | | |

Conclusion: Nose-to-brain delivery formulation of nimodipine ST202-Y-4 was administered to rats at a dose 1/10 that of oral control ST-1, the plasma, cerebrospinal fluid and brain drug exposures were 5.11, 4.89 and 9.78 times higher than that of oral control ST-1, and the peak plasma, cerebrospinal fluid, and brain concentrations were 5.34, 2.35, and 11.54 times higher than that of oral control ST-1, respectively.

Tissue distribution in rats, brain>plasma>cerebrospinal fluid, and the exposure ratio of brain/plasma was higher for the nasal brain delivery of nimodipine, ST202-Y-4, than for the oral control, ST-1 (2.47 vs 1.29), and the peak ratio was higher than that of the oral control, ST-1 (2.30 vs 1.07).

At the terminal time point of 120 min, nose-to-brain delivery of nimodipine, ST202-Y-4, was distributed in tissues at concentrations ranging from 20.83% to 100.00% of peak, whereas oral control ST-1 had been massively eliminated to 7.33% to 14.33% of peak.

### Pharmacologic Embodiment 4:14-Day Rat Toxicology Pre-Test

The saline, full-excipient, high-dose and low-dose nasal brain delivery formulations were administered intranasally to four rats of each sex in each group, and validated after 14 days of administration, exemplarily, in this pharmacological experimental example:

The full-excipients and ST-202-Y-High, using the grouping ratios shown in Table 17 below, which can be prepared by the methods disclosed in Example 1 in this application or by methods known to those skilled in the art;

TABLE 17

| Formulation | full-excipient | ST202-Y-High |
|---|---|---|
| Nimodipine | NA* | 7.5 |
| Transcutol P | 48.0 | 60 |
| Lecithin | 40.0 | 50 |
| PL-100M | | |
| ethyl oleate | 311.0 | 381.25 |
| grape essence | 1.0 | 1.25 |
| Total content | 400 | 500.0 |

NA* represent 0, and mesns it does not contain nimodipine.

ST202-Y-Low, the formulation was prepared at a concentration of 5 mg/g. The formulation was prepared by weighing 120 g of the full excipient with 60 g of the formulation of ST202-Y-High and mixing to obtain.

The experimental groupings are shown in Table 18 below,

TABLE 18

| | | | | Administra- |
|---|---|---|---|---|
| | Dosage | Dosage | Formulation | tion volume |
| | (mg/kg/ | (mg/kg/ | concentration | (mL/kg/ |
| group | dose) | day) | (mg/g) | dose) |
| saline | — | — | — | 0.56 |
| full-excipient | — | — | — | 0.56 |
| ST202-Y-Low | 2.5 | 5 | 5 | 0.56 |
| ST202-Y-High | 7.5 | 15 | 15 | 0.56 |

The experimental monitoring items and their results are as follows:

### 1. Cage-Side Observation,

No abnormality in the appearance, behavioral activities, mental status, respiratory status, hair status and other general status of the rats were observed during the experiment.

### 2. Weight Gain was as Shown in Table 19 Below,

TABLE 19
Group

| (n = 8, male and | Dosage | 1-14 days weight gain | | | |
|---|---|---|---|---|---|
| female each half) | (mg/kg/day) | Male | | Female | |
| saline | — | 97 g | — | 37 g | — |
| full-excipient | — | 90 g | −7% | 33 g | −11% |
| ST202-Y-Low | 5 | 97 g | 0% | 30 g | −18% |
| ST202-Y-High | 15 | 87 g | −11% | 35 g | −5% |

Body weight gain CONCLUSION: There was no abnormality in the body weight gain of rats in each dose group.

3. Changes in organ weights are shown in Table 20 below:

TABLE 20

| | | | | | | | |
|---|---|---|---|---|---|---|---|
| Group | | | | | | | |
| (n = 8, male and | dosage | | | | | | |
| female each half) | (mg/kg/day) | brain | hearts | liver | spleen | lungs | kidney |

male

| | | | | | | | |
|---|---|---|---|---|---|---|---|
| full-excipient | | −2% | 6% | −4% | −11% | −11% | 0 |
| ST202-Y-Low | 5 | −2% | 0% | −9% | 3% | −4% | −3% |
| ST202-Y-High | 15 | −2% | −7% | −10% | −10% | 26% | −6% |

female

| | | | | | | | |
|---|---|---|---|---|---|---|---|
| full-excipient | | −11% | −1% | −4% | 0 | −4% | 3% |
| ST202-Y-Low | 5 | −6% | −1% | −8% | −1% | 4% | −4% |
| ST202-Y-High | 15 | −4% | 0 | −12% | −4% | 3% | −5% |

Changes in organ weights CONCLUSION: There was no abnormality in the organ weights of the rats in each dose group.

### 4. Nasal Irritation Observations:

The appearance of the nose was observed in pairs for 30 min before and after the administration of the drug and recorded by photographs or videos, and expressed as points according to the “Grading Criteria for Local Mucosal Irritation Response”. The average score of 0.00-0.40 was no irritation; the average score of 0.41-1.50 was mild irritation; the average score of 1.51-2.50 was moderate irritation; the average score of >2.51 was severe irritation.

Evaluation of mucosal irritation: the mucosal irritation response of rats in each dose group was graded as no irritation; as shown in Table 21 below.

TABLE 21

| | Irritation score | | | | Grade of mucosal | |
|---|---|---|---|---|---|---|
| | Before administration | | After administration | | irritation response | |
| Group n = 8 | Male | Female | Male | Female | Male | Female |
| saline | 0 | 0 | 0 | 0 | No irritation | No irritation |
| full-excipient | 0 | 0 | 0.07 | 0.02 | No irritation | No irritation |
| ST202-Y-Low | 0 | 0 | 0.09 | 0.04 | No irritation | No irritation |
| ST202-Y-High | 0 | 0 | 0.18 | 0.07 | No irritation | No irritation |

### 5. Pathology Results

After 14 days of administration, pathological sections of rats were taken, and the experimental results revealed that: there was no difference in the amount of drug exposure between male and female animals in this experiment, and there was no difference in the morphology of cases between males and females; there were no pathological changes in the olfactory bulb of the brain or in other brain regions in all groups; no pathological changes were found in the mucosa of the turbinate region and the olfactory region of the nasal area in all groups; and, in conclusion: the full-excipient and the formulation did not show any toxicity to the rats.

Obviously, the above embodiments are merely examples for the purpose of clear illustration, and are not a limitation of the embodiments. For those of ordinary skill in the art, other variations or changes in different forms may be made on the basis of the above description. It is neither necessary nor possible to exhaust all of the embodiments herein. The obvious variations or changes derived therefrom are still within the scope of protection of the present application.

1. A nose-to-brain delivery formulation of nimodipine, characterized in that, the nose-to-brain delivery formulation comprising nimodipine, a solvent, a diluent and an absorption enhancer, wherein the solvent is diethylene glycol monoethyl ether, and the absorption enhancer is one or more substances selected from phospholipids, n-dodecyl-β-D-maltoside, cyclopentadecanolide, Polyoxyl 15 Hydroxystearate, and oleoyl polyoxyl-6 glycerides.

2. The nose-to-brain delivery formulation of nimodipine of claim 1, characterized in that, the diluent can be one or more substances selected from ethyl oleate, isopropyl myristate, medium chain triglycerides, castor oil, soybean oil, sesame oil, oleic acid, vitamin E, glyceryl monolinoleate, propylene glycol laurate, or propylene glycol dicaprylate/dicaprate.

3. The nose-to-brain delivery formulation of nimodipine of claim 1, characterized in that, the content of the nimodipine is 4-50 parts by weight; the content of the solvent is 5-300 parts by weight; the content of the diluent is 500-900 parts by weight; and the content of the absorption enhancer is 20-200 parts by weight.

4. The nose-to-brain delivery formulation of nimodipine of claim 3, characterized in that, the content of the nimodipine is 5-40 parts by weight; the content of the solvent is 40-200 parts by weight; the content of the diluent is 700-850 parts by weight; and the content of the absorption enhancer is 40-160 parts by weight.

5. The nose-to-brain delivery formulation of nimodipine of claim 1, characterized in that, the phospholipids is lecithin or soya lecithin.

6. The nose-to-brain delivery formulation of nimodipine of claim 1, characterized in that, the lecithin is lecithin E80 or lecithin PL100M, and the soya lecithin is soya lecithin S75 or soya lecithin S100.

7. The nose-to-brain delivery formulation of nimodipine of claim 1, characterized in that, the nose-to-brain delivery formulation of nimodipine further contains 0-50 parts by weight aromatizer.

8. The nose-to-brain delivery formulation of nimodipine of claim 7, characterized in that, the aromatizer is grape essence.

9. The nose-to-brain delivery formulation of nimodipine of claim 1, characterized in that, the nose-to-brain delivery formulation of nimodipine contains 5-40 parts by weight nimodipine, 40-200 parts by weight diethylene glycol monoethyl ether, 40-160 parts by weight phospholipids, 700-850 parts by weight ethyl oleate.

10. The nose-to-brain delivery formulation of nimodipine of claim 9, characterized in that, the nose-to-brain delivery formulation of nimodipine contains 15 parts by weight nimodipine, 100 parts by weight diethylene glycol monoethyl ether, 100 parts by weight phospholipids, 795 parts by weight ethyl oleate.

11. The nose-to-brain delivery formulation of nimodipine of claim 1, characterized in that, the nose-to-brain delivery formulation of nimodipine acts as drug reservoirs.

12. A method for preventing or treating an ischemic disorder in a patient comprising administering to a subject in need thereof a therapeutically effective amount of a nose-to-brain delivery formulation of nimodipine, the nose-to-brain delivery formulation comprising nimodipine, a solvent, a diluent and an absorption enhancer, wherein the solvent is diethylene glycol monoethyl ether, and the absorption enhancer is one or more substances selected from phospholipids, n-dodecyl-β-D-maltoside, cyclopentadecanolide, Polyoxyl 15 Hydroxystearate, and oleoyl polyoxyl-6 glycerides.

13. The method of claim 12, the ischemic disorder is selected from cerebral ischemic disorders, cerebral vasospasm caused by subarachnoid hemorrhage, and sudden deafness.

14. A composition, comprising a nose-to-brain delivery formulation of nimodipine and pharmaceutically acceptable excipient, the nose-to-brain delivery formulation comprising nimodipine, a solvent, a diluent and an absorption enhancer, wherein the solvent is diethylene glycol monoethyl ether, and the absorption enhancer is one or more substances selected from phospholipids, n-dodecyl-β-D-maltoside, cyclopentadecanolide, Polyoxyl 15 Hydroxystearate, and oleoyl polyoxyl-6 glycerides.
